(19) United States
(12) Patent Application Publication
(10) Pub. No.: US 2022/0331971 A1
(43) Pub. Date: Oct. 20, 2022

(54) SYSTEMS, TARGETS, AND METHODS FOR OPTICAL REGISTRATION OF TOOLS

(71) Applicant: Intellijoint Surgical Inc., Kitchener (CA)

(72) Inventors: JOSEPH ARTHUR SCHIPPER, KITCHENER (CA); HANNAH SARA ROSENBERG, TORONTO (CA); SAMANTHA MCCABE, KITCHENER (CA)

(21) Appl. No.: 17/716,600

(22) Filed: Apr. 8, 2022

Related U.S. Application Data
(60) Provisional application No. 63/175,722, filed on Apr. 16, 2021.

(51) Int. Cl. B25J 9/16; B25J 13/08; G06T 7/73; G06T 7/246

(52) U.S. Cl. CPC B25J 9/1697; B25J 13/08; G06T 7/75; G06T 7/251; A61B 17/1613

(57) ABSTRACT

Described are systems, targets, and methods for registering a tool for use in optical tracking. A first target and a second target are attached to the tool, with the first target having a known spatial relationship to the tool or end effector of the tool. By determining a spatial feature of the first target and a pose of the second target, and using the known spatial relationship between the first target and the tool or end effector of the tool, a spatial relationship between the second target and the end effector can be determined. Subsequently the first target can be removed, and the end effector is trackable based on only tracking of the second target. In some implementations, the first target is removably couplable to the tool by the same interface by which the end effector is removably couplable to the tool.

### CROSS-REFERENCE

The present application claims a domestic benefit of U.S. Provisional Application No. 63/175,722, filed Apr. 16, 2021, the entire contents of which are incorporated herein by reference.

### FIELD OF THE INVENTION

The present disclosure relates to registration of tools, and in particular relates to systems and targets for optically registering tools for optical tracking, and methods for performing such optical registration.

### BACKGROUND

During a procedure, such as a surgical procedure, it can be desirable to register, detect, localize, and/or track various elements. Such elements can include, for example, tools used during the surgery. Optical tracking typically entails positioning a target on the element to be tracked, capturing image data representing the target, and determining a pose (position and orientation) of the target, or of the tool relative to the target by a tracking system. Accurate tracking of the tool requires that the tracking system knows important spatial features of the tool relative to the target. One way to provide this information to the tracking system is to register the tool and tracker, to which the present disclosure is directed.

The targets and methods described herein are not limited to surgical applications, but rather can be used in any appropriate application.

### SUMMARY

Described are systems, targets, and methods for registering a tool for use in optical tracking. A first target and a second target are attached to the tool, with the first target having a known spatial relationship to the tool or end effector of the tool. By determining a spatial feature of the first target and a pose of the second target, and using the known spatial relationship between the first target and the tool or end effector of the tool, a spatial relationship between the second target and the end effector can be determined. Subsequently the first target can be removed, and the end effector is trackable based on only tracking of the second target. In some implementations, the first target is removably couplable to the tool by the same interface by which the end effector is removably couplable to the tool.

According to a broad aspect, the present disclosure describes a system for registering a tool for tracking of said tool, the tool comprising a first end for use with an end effector, the system comprising: an image sensor; a first target removably couplable to the first end of the tool with a known spatial relationship to the end effector, the first target being optically detectable to the image sensor; a second target couplable to the tool spatially separate from the first target, the second target being optically detectable to the image sensor; and a processing unit configured to: receive image data from the image sensor, the image data including representations of the first target and the second target with which to determine a spatial feature of the first target and determine a pose of the second target; determine a spatial relationship between the second target and a spatial feature of the end effector, based on a spatial relationship determined between the spatial feature of the first target and the pose of the second target, and based on the known spatial relationship between the first target and the end effector; and provide the spatial relationship between the second target and the spatial feature of the end effector, for subsequent tracking of the end effector based on the second target.

The first target may comprise an optically detectable planar surface. The first target may comprise an optically detectable planar disk. The first target may comprise at least a first surface and a second surface adjacent the first surface, the first surface being a planar surface which is optically detectable relative to the second surface. The first target may have a cylindrical shape, the first surface being a planar end surface of the cylindrical shape, the second surface being a curved surface adjacent the planar end surface.

The second target may comprise a plurality of optically detectable markers coupled to a support, the support removably couplable to the tool.

The first target may be removably couplable to the tool concurrently with the end effector coupled to the first end of the tool. The system may further comprise an interface to removably couple the first target to the end effector. The interface may be integral with the first target.

The end effector may be removably couplable to the first end of the tool by an interface, and the first target may be removably couplable to the first end of the tool by the interface with the end effector absent.

The tool may be a reamer comprising a shaft and a reamer head, the reamer head being the end effector and comprising a first interface portion, the shaft comprising a second interface portion removably couplable to the first interface portion of the reamer head; the first target may comprise a third interface portion removably couplable to the second interface portion of the shaft; and the known spatial relationship between the first target and the end effector may comprise a known offset between a center of the first target when removably coupled to the shaft and a center of rotation of the reamer head.

The tool may be a cup impactor comprising a shaft and a cup, the cup being the end effector and comprising a first interface portion, the shaft comprising a second interface portion removably couplable to the first interface portion of the cup; the first target may comprise a third interface portion removably couplable to the second interface portion of the shaft; and the known spatial relationship between the first target and the end effector may comprise a known offset between a center of the first target when removably coupled to the shaft and a center of rotation of the cup.

The tool may be one of a plurality of candidate tools, and the first target may comprise a plurality of interface portions, each interface portion configured to removably couple to a cooperating interface portion on at least one tool of the plurality of candidate tools. The plurality of interface portions may comprise a first interface portion and a second interface portion, the first interface portion positioned on a first side of the first target, the second interface portion positioned on a second side of the first target opposite the first side. The plurality of interface portions may comprise at least a first interface portion and a second interface portion, the first interface portion and the second interface portion positioned on a first side of the first target, and at least one optically detectable region may be positioned on a second side of the first target opposite the first side. A first optically detectable region may be positioned on the second side of the first target opposite the first interface portion; and a second optically detectable region may be positioned on the second side of the first target opposite the second interface portion. The first optically detectable region may comprise an optically detectable pattern distinct from the second optically detectable region.

The tool may be one of a plurality of candidate tools, and the system may further comprise a plurality of adapters, each adapter comprising a respective interface portion for coupling to a cooperating interface portion of at least one tool of the plurality of candidate tools, the first target comprising an adapter coupler for coupling to each adapter of the plurality of adapters.

The first target may comprise a circular optically detectable region, and the processing unit being configured to determine a spatial feature of the first target may comprise the processing unit being configured to: identify a periphery of the optically detectable region as represented in the image data; model a plurality of rays extending from the image sensor to the periphery of the optically detectable region; and fit a circle to the model of the plurality of rays.

The processing unit being configured to receive image data from the image sensor may comprise the processing unit being configured to receive a plurality of images from the image sensor, each of the plurality of images including a representation of the first target and a representation of the second target as viewed from different positions. The first target may comprise a circular optically detectable region, and the processing unit being configured to determine a spatial feature of the first target may comprise the processing unit being configured to: identify a periphery of the optically detectable region as represented in each of the plurality of images; model a plurality of rays extending from the image sensor to the periphery of the optically detectable region for each image of the plurality of images; and fit a circle to the model of the plurality of rays for the union of the plurality of images.

The first target may comprise an optically detectable planar surface having a rotationally asymmetric shape; and the processing unit may be further configured to determine orientation of the first target about an axis perpendicular to the optically detectable planar surface based on a shape of the optically detectable planar surface as represented in the image data.

The first target may comprise a planar surface having a rotationally asymmetric optically detectable pattern thereon; and the processing unit may be further configured to determine orientation of the first target about an axis perpendicular to the planar surface based on the optically detectable pattern as represented in the image data. The optically detectable pattern may comprise at least one optically detectable region extending radially from a center of the planar surface to a periphery of the planar surface. The optically detectable pattern may comprise at least one first region which appears to the image sensor with a first brightness and at least one second region which appears to the image sensor with a second brightness lower than the first brightness, wherein the second region is positioned spatially non-centered on the planar surface of the first target.

The system may further comprise a non-transitory processor readable storage medium, and the processing unit being configured to provide the spatial relationship between the second target and the spatial feature of the end effector may comprise: the processing unit being configured to provide the spatial relationship between the second target and the spatial feature of the end effector to the non-transitory processor-readable storage medium for storage and subsequent access.

The system may further comprise a non-transitory processor-readable storage medium having a model of the first target stored thereon, the processing unit further configured to receive the model of the first target from the non-transitory processor-readable storage medium, with which to determine the spatial feature of the first target based on the image data.

The system may further comprise a non-transitory processor-readable storage medium having a model of the second target stored thereon, and the processing unit may be further configured to receive the model of the second target from the non-transitory processor-readable storage medium, with which to determine the pose of the second target based on the image data.

According to another broad aspect, the present disclosure describes: a computer-implemented method of registering a tool for tracking of said tool, the method comprising: receiving image data from an image sensor, the image data including: a representation of a first optically detectable target removably coupled to a first end of the tool with a known spatial relationship to an end effector of the tool, with which to determine a spatial feature of the first target; and a representation of a second optically detectable target coupled to the tool spatially separate from the first target, with which to determine a pose of the second target; determining a spatial relationship between the second target and a spatial feature of the end effector, based on a spatial relationship determined between the spatial feature of the first target and the pose of the second target, and based on the known spatial relationship between the first target and the end effector; and providing the spatial relationship between the second target and the spatial feature of the end effector, for subsequent tracking of the end effector based on the second target.

The first target may comprise a circular optically detectable region, and determining a spatial feature of the first target may comprise: identifying a periphery of the optically detectable region as represented in the image data; modeling a plurality of rays extending from the image sensor to the periphery of the optically detectable region; and fitting a circle to the model of the plurality of rays.

Receiving image data from the image sensor may comprise receiving a plurality of images from the image sensor, each of the plurality of images including a representation of the first target and a representation of the second target. The first target may comprise a circular optically detectable region, and determining a spatial feature of the first target may comprise: identifying a periphery of the optically detectable region as represented in each of the plurality of images; modeling a plurality of rays extending from the image sensor to the periphery of the optically detectable region for each image of the plurality of images; fitting a circle to the model of the plurality of rays for the union of the plurality of images.

Providing the spatial relationship between the second target and the spatial feature of the end effector may comprise: providing the spatial relationship between the second target and the spatial feature of the end effector to a non-transitory processor-readable storage medium for storage and subsequent access.

The method may further comprise retrieving a model of the first target from a non-transitory processor-readable storage medium, with which to determine the spatial feature of the first target based on the image data.

The method may further comprise retrieving a model of the second target from a non-transitory processor-readable storage medium, with which to determine the pose of the second target based on the image data.

According to yet another broad aspect, the present disclosure describes a target for use in registering a tool for optical tracking by a tracking system, the tool comprising a first end having a first interface portion for removably coupling to a second interface portion of an end effector, the target comprising: a planar first surface, at least a region of the first surface optically detectable by the tracking system; a third interface portion configured to removably couple the target to the first interface portion of the tool with the end effector absent, the target removably couplable to the tool with a known spatial relationship between the target and the tool.

The first surface may comprise an optically detectable circular planar disk.

The target may further comprise a second surface adjacent the first surface, the first surface being optically detectable relative to the second surface. The target may have a cylindrical shape, the first surface being a planar end surface of the cylindrical shape, the second surface being a curved surface adjacent the planar end surface.

The tool may be a reamer comprising a shaft and a reamer head, the reamer head being the end effector and comprising the second interface portion, the shaft comprising the first interface portion for removably coupling to the second interface portion of the reamer head; the third interface portion of the target may be removably couplable to the first interface portion of the shaft; and the known spatial relationship between the target and the tool may comprise a known offset between a center of the first interface portion and a center of the first surface of the target when the target is removably coupled to the shaft.

The tool may be a cup impactor comprising a shaft and a cup, the cup being the end effector and comprising the second interface portion, the shaft comprising the first interface portion for removably coupling to the second interface portion of the cup; the third interface portion of the target is removably couplable to the first interface portion of the shaft; and the known spatial relationship between the target and the tool may comprise a known offset between a center of the first interface portion and a center of the first surface of the target when the target is removably coupled to the shaft.

The target may further comprise a fourth interface portion, the fourth interface portion configured to removably couple to another tool having a fifth interface portion. The third interface portion may be positioned on a first side of the target, and the fourth interface portion may be positioned on a second side of the target opposite the first side. The third interface portion and the fourth interface portion may be positioned on a first side of the target, and at least one optically detectable region may be positioned on a second side of the target opposite the first side. A first optically detectable region may be positioned on the second side of the target opposite the third interface portion; and a second optically detectable region may be positioned on the second side of the target opposite the fourth interface portion. The first optically detectable region may comprise an optically detectable pattern distinct from the second optically detectable region.

The target may further comprise an adapter coupler portion for coupling to each of a plurality of adapters, wherein the target is removably couplable to a plurality of candidate tools by respective adapters of the plurality of adapters, and wherein the third interface portion is comprised by one of said plurality of adapters.

The planar first surface may have a rotationally asymmetric shape, to indicate to the tracking system orientation of the target about an axis perpendicular to the planar first surface. The planar first surface may have a rotationally asymmetric optically detectable pattern thereon, to indicate to the tracking system orientation of the target about an axis perpendicular to the planar first surface. The optically detectable pattern may comprise at least one optically detectable region extending radially from a center of the first surface to a periphery of the first surface. The optically detectable pattern may comprise at least one first region which appears with a first brightness and at least one second region which appears with a second brightness lower than the first brightness, wherein the second region is positioned spatially non-centered on the planar surface of the target.

### BRIEF DESCRIPTION OF THE FIGURES

FIG. 1 illustrates an exemplary scenario in which a tracking system is used.

FIG. 2A is a side partial cutaway view of an exemplary tool having a target and an end effector coupled thereto. FIG. 2B is a side view of the tool shown in FIG. 2A, with another target coupled in place of the end effector. FIG. 2C is an enlarged side view of the tool and target shown in FIG. 2B. FIG. 2D is an enlarged side view of the tool and end effector shown in FIG. 2A. FIG. 2E is a side view of an exemplary tool having two targets and an end effector coupled thereto.

FIG. 3A is a front view of an exemplary target. FIG. 3B is an isometric view of the target shown in FIG. 3A. FIGS. 3C, 3D, 3E, 3F, 3G, and 3H are front views of alternative exemplary targets.

FIG. 4A is a rear view of an exemplary reamer head end effector, and an interface portion by which the reamer head couples to a shaft. FIG. 4B is a side cross-sectional view of the reamer head of FIG. 4A, and a shaft to which the reamer head is to couple. FIG. 4C is a side cross-sectional view of the reamer head and shaft of FIG. 4B coupled together.

FIG. 5A is a side cross-sectional view of a target, and a shaft to which the target is to couple, via an interface similar to a reamer coupling interface. FIG. 5B is a side cross-sectional view of the target and shaft of FIG. 5A coupled together.

FIG. 6A is a front view of an exemplary target removably couplable to a tool while an end effector is coupled to the tool. FIG. 6B is a side cross-sectional view of the target of FIG. 6A, and a tool to which the target is to be coupled. FIG. 6C is a side cross-sectional view of the target and tool of FIG. 6B coupled together.

FIG. 7 is a side cross-sectional view of a target and a tool, and an adapter by which the target is coupled to the tool.

FIG. 8A is a rear view of an exemplary cup end effector, and an interface portion by which the cup couples to a shaft. FIG. 8B is a side cross-sectional view of the cup of FIG. 8A, and a shaft to which the cup is to couple. FIG. 8C is a side cross-sectional view of the cup and shaft of FIG. 8B coupled together.

FIG. 9A is a side cross-sectional view of a target, and a shaft to which the target is to couple, via an interface similar to a cup coupling interface. FIG. 9B is a side cross-sectional view of the target and shaft of FIG. 9A coupled together. FIG. 9C is a side cross-sectional view of the target and shaft coupled together as in FIG. 9B, showing spatial features of the target and shaft.

FIG. 9D is a side cross-sectional view of the cup and shaft coupled together as in FIG. 8C, showing spatial features of the cup and shaft.

FIG. 10A is a side cross-sectional view of a target having a plurality of interface portions for coupling to different tools. FIG. 10B is a side cross-sectional view, and FIG. 100 is a front view, of a target having a plurality of interface portions and a plurality of optically detectable regions. FIG. 10D is a side cross-sectional view of a target having an adapter coupler region for receiving adapters for coupling different tools to the target.

FIG. 11 is a flowchart diagram illustrating a method of registering a tool from the perspective of an operator.

FIG. 12 is a flowchart diagram illustrating a method of registering a tool from the perspective of a tracking system.

FIG. 13 is a ray model diagram showing a model of rays extending from a target identified in image data.

### DETAILED DESCRIPTION

The description herein details several exemplary embodiments. One skilled in the art will appreciate that it is within the scope of the present disclosure to combine individual embodiments with other embodiments as appropriate.

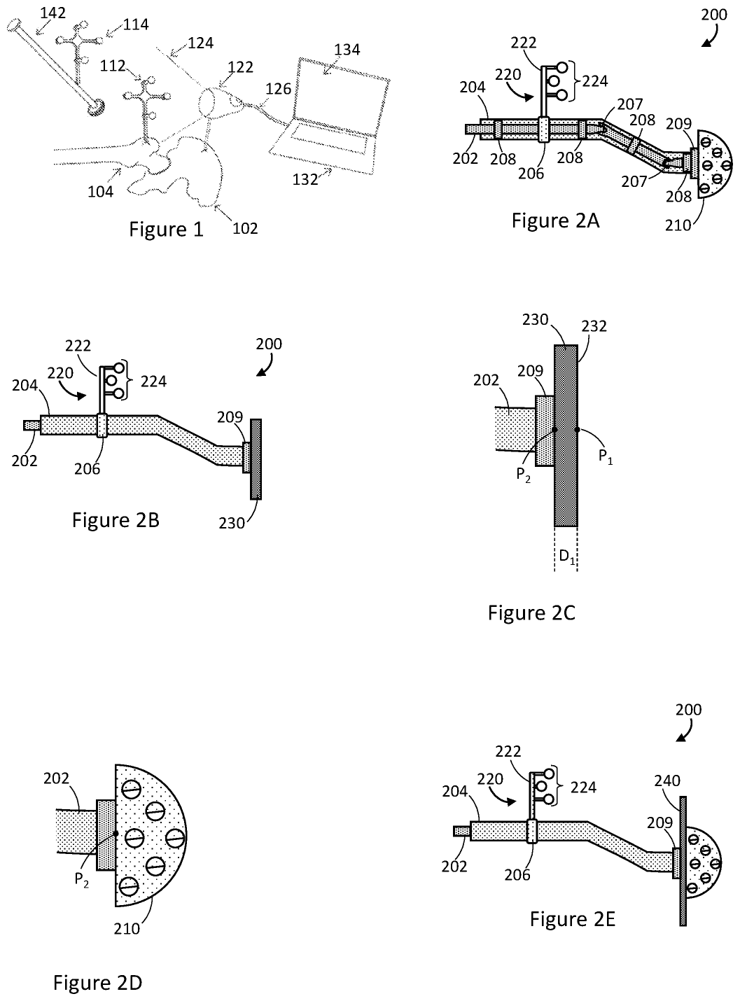

FIG. 1 illustrates an exemplary scenario in which a surgical procedure is being performed. Any of the targets, tracking systems, or methods described herein can be used in or in support of the context described with reference to FIG. 1, and in the ways described with reference to FIG. 1.

In the example of FIG. 1, a total hip arthroplasty (THA) is being performed, but the discussion herein is applicable to registration for any surgical procedure where a tracking system is used, or any appropriate procedure other than surgery. In FIG. 1, a patient's pelvis 102 and femur 104 are shown. A target 112 is positioned on (e.g. affixed to, mounted on, or touched against) femur 104. An image sensor 122 is positioned on pelvis 102, though for registration steps image sensor 122 could be placed on a surface, held in the hands of an operator, coupled to a mount, or any other appropriate positions. Image sensor 122 can capture image data over a field of view 124. Image sensor 122 can communicate captured image data to computing device 132. Image sensor 122 is shown as being communicatively coupled to computing device 132 by wire 126, but wireless communication between image sensor 122 and computing device 132 is also possible. Further, it is also possible for image sensor 122 and computing device 132 to be a unified device. Computing device 132 can analyze the image data (for example by at least one processing unit in computing device 132), or computing device 132 can send the data to a remote device or cloud server for analysis by a processing unit thereon, to detect target 112 and determine a pose (position and orientation) thereof. Pose can be position or orientation in three-dimensional space, though in certain applications pose can be position and orientation in two-dimensional space. Further, based on the pose and pre-determined geometry of target 112, computing device 132 can also determine a pose of elements which target 112 is positioned on. In the example of FIG. 1, image sensor 122 can be affixed to pelvis 102, and target 112 can be affixed to femur 104. Consequently, movement of target 112 relative to image sensor 122 can correspond to movement of the femur 104 relative to pelvis 104. In this context, “tracking” an element can entail continuously, regularly, or intermittently determining a pose of the element.

FIG. 1 also illustrates target 114 positioned on a tool 142. In the case of FIG. 1, tool 142 is a cup impactor for implanting a prosthetic hip cup during THA, but target 114 can be positioned on any appropriate tool. As examples, target 114 could be coupled to the tool by clips or fasteners; or target 114 could be removably coupled to the tool by magnetism (directly, or indirectly via a magnetic mount secured to the tool). Image sensor 122 can capture image data including target 114, which can subsequently be analyzed by computing device 132 (or a remote analysis device as mentioned above) to determine pose information of tool 142. Target 114 can be identical to target 112, or target 114 and target 112 could be different (for example by having different geometry from each other). In some implementations, target 112 could be removably positioned on a base mounted to femur 104, such that target 112 can be removed from and replaced on femur 104 without affecting the positioning of target 112 when positioned on femur 104. In such cases, target 112 can be removed from the base, and positioned on other elements (such as tool 142), such that multiple tracking operations can be achieved with a single target. In such implementations, the functionality of target 114 could be achieved with target 112.

Information based on the pose of an element of interest can be presented by display 134 of computing device 132 (or another device). This information can provide helpful or critical information to the surgeon. Further, other output means can also be used, such as audio output like speakers.

In order to accurately determine the pose of an end effector of tool 142, geometry of the tool relative to target 114 should be known to the tracking system. This could be achieved by precise manufacturing of tool 142 with specific geometry, which is provided to the tracking system. Alternatively, for generic tools or tools where precise geometric information is not available to the tracking system, registration or calibration steps can be performed to determine the geometry of the tool relative to target 114. In the example of FIG. 1, registration can determine a spatial relationship between the cup on tool 142 (the end effector) and target 114, so that the cup can be accurately positioned by tracking target 114. Systems, targets, and methods for such registration are discussed herein.

FIG. 1 shows exemplary anatomy of pelvis 102 and femur 104. However, the present disclosure is applicable to surgeries pertaining to any appropriate anatomy, including for example leg, arm, torso, head, back, or chest anatomy, including bones therein. As mentioned above, the targets discussed herein can also be used in non-surgical applications.

Throughout this disclosure, reference is made to a “tracking system”. Such a tracking system can refer to a device such as computing device 132, or any other appropriate device capable of processing, which can receive data representing a target, and determining a pose of the target or pose of an element in contact with the target. Broadly, a tracking system can also include an image sensor and a target.

FIG. 2A is a side partial cutaway view, and FIGS. 2B, 2C, 2D, and 2E are side views, of a tool 200. In the example of FIGS. 2A-2E, the tool 200 is an acetabular reamer, though the discussion applies to other tool types, and other acetabular reamers shapes and styles, as is detailed later. Tool 200 comprises a shaft 202 having a first end with a reamer head 210 (an end effector) coupled thereto by an interface 209. Shaft 202 has a second end opposite the first end. Shaft 202 is positioned at least partially within a sheath 204. FIG. 2A is a partial cutaway view in that a portion of sheath 204 which would cover shaft 202 from the view is not illustrated. FIGS. 2B-2E are side views of tool 200, but not cutaway views. Sheath 204 is illustrated as being a sleeve over shaft 202, and can act as a handle by which an operator can grab tool 200. Additionally, other forms of handle, such as a protrusion which extends away from sheath 204, could be used. Shaft 202 is shown as having an offset shape (two portions along different axes connected by an intermediate portion), each portion connected by a joint 207, but shafts with a continuous straight shape could also be used. For such straight shafts, sheath 204 can be smaller, running along only a small portion of the shaft to act as a grip for an operator. The second end of shaft 202 opposite interface 209 is shown protruding from sheath 204, and can couple to an actuator or actuator interface for rotating shaft 202 in sheath 204. Joints 207 can be universal joints, or other types of joints which impart rotation from one portion of shaft 202 to another portion of shaft 202 at a different angle, to cause rotation of all portions of the shaft 202. Spacers 208 keep shaft 202 aligned within sheath 204, while enabling rotation of shaft 202 without imparting rotation to sheath 202. Spacers 208 can be bearings, for example. Rotation of shaft 202 imparts rotation to reamer head 210, for reaming of an acetabulum.

A target 220 is coupled to sheath 204. The exemplary target 220 as shown in FIGS. 2A, 2B, and 2E includes a support structure 222, and a plurality of optically detectable markers 224 coupled to the support structure. However, other target constructions are possible. In use, a tracking system identifies a pose of target 220. In some implementations, the tracking system compares the position of patterns, features, or shapes of the target represented in the image data (e.g. positions of markers 224) to positions of the patterns, features, or shapes in a model of the target. For example, a CAD or computer model of the target could be accessed by the tracking system, the model having a geometry which matches a known geometry of the target. In other examples, the model could be a simplified model which indicates relative orientation and position of important patterns, features, or shapes, and a key point of the target (such as the tip of an extension from the target, or a centroid of the target).

Throughout this disclosure, “optically detectable” refers to detectability by a tracking system, and does not necessarily correspond to what is seen by the human eye. For example, an optically detectable pattern could be visible in the infrared spectrum outside of the normal wavelength range of human vision. Generally, optical detectability entails a contrast between at least two regions or components. In some implementations, an optically detectable element comprises regions or components of different color. In other implementations, an optically detectable element comprises regions or components which appear to a tracking system as “bright” and other regions or components which appear to a tracking system as “dark”. “Bright” and “dark” as used herein are relative to each other, such that a “bright” region or material appears to a tracking system with greater brightness than a “dark” region or material. Exemplary “bright” materials can include reflective, retroreflective, back-illuminated, or light colored (e.g. white) materials. Exemplary “dark” materials can include opaque, non-reflective, light-absorptive, or dark colored (e.g. black) materials. In cases where a “bright” material is a reflective or retroreflective material, an image sensor in a tracking system can be proximate to or constructed in combination with at least one light source to illuminate the material.

In the exemplary target 220 shown in FIGS. 2A, 2B, and 2E, markers 224 are made of or coated with a “bright” material, so as to be detectable relative to an environment of tracker 220. Support structure 222 is made or coated with a “dark” material so as to not interfere with detection of markers 224 by a tracking system.

The target 220 can be removably coupled to the sheath 204, such as via a releasable clamp or magnetic mount mechanism 206. Alternatively, the target 220 can be permanently fixed to the sheath 204. Image data is captured by an image sensor of a tracking system, the image data including a representation of target 220. From this image data, the pose of target 220 is determined, and in turn pose of the tool 200 is determinable. Registration between tool 200 and target 220 establishes a spatial relationship between components of tool 200 (such as the end effector: reamer head 210) and target 220. Thus, by performing such registration, spatial features of the reamer head 210 can be determined based on pose of the target 220. For example, reaming depth of an acetabulum can be determined by comparing a center of rotation in the acetabulum prior to reaming, and the center of rotation of reamer head 210 during or after reaming.

FIGS. 2B-2D illustrate an exemplary implementation for registration to determine a spatial relationship between at least one spatial feature of an end effector and target 220.

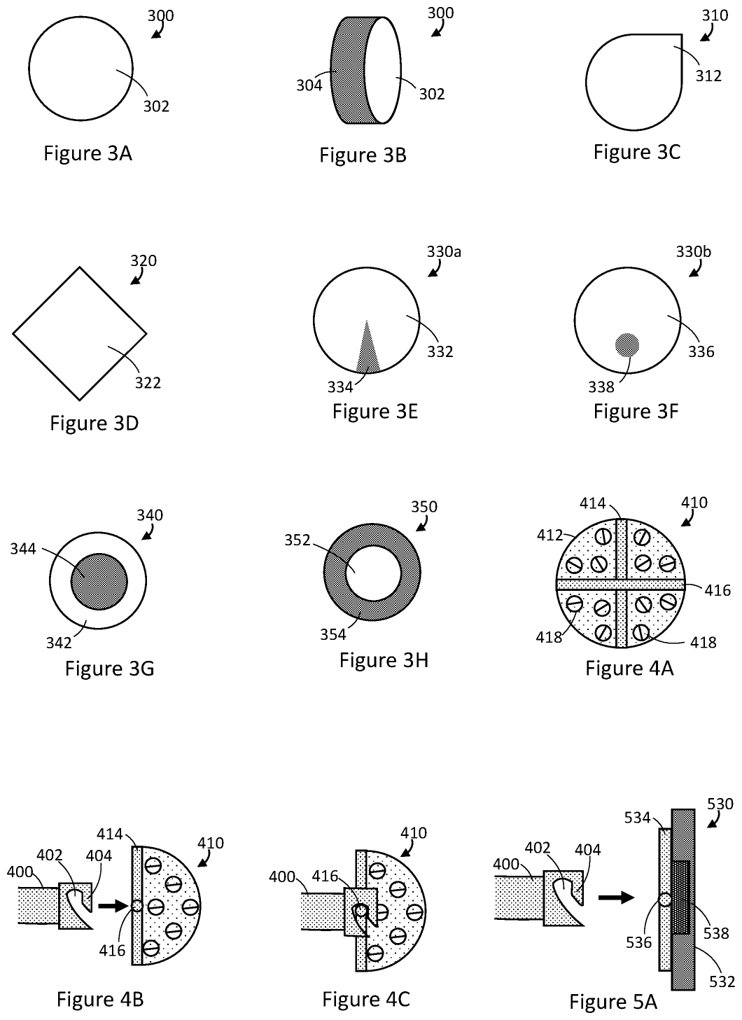

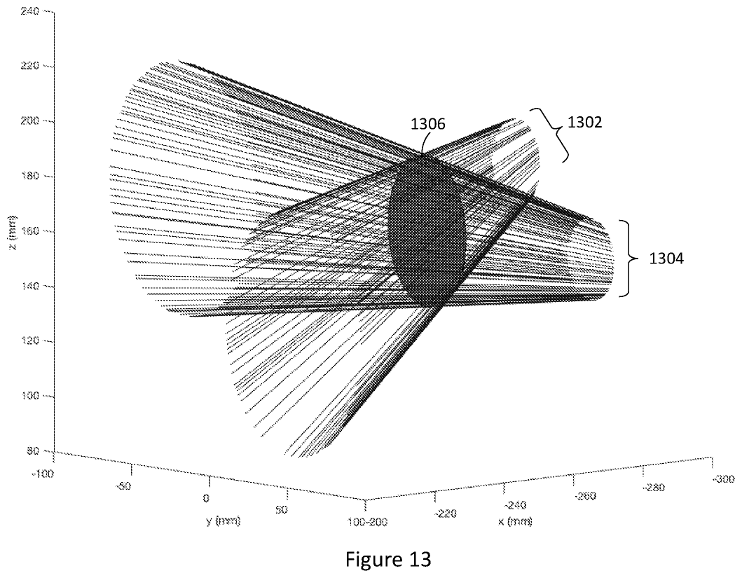

FIG. 2B shows tool 200 with reamer head 210 absent. That is, a coupling between shaft 202 and reamer head 210 is a removable coupling, and reamer head 210 is detached from shaft 202 (or wasn't attached in the first place). In place of reamer head 210, a target 230 is attached to the first end of shaft 202. Exemplary interfaces for coupling are described later with reference to FIGS. 4A-4C, 5A-5B, 8A-8C, 9A-9D, and 10. The target 230 is optically detectable by a tracking system for registration of tool 200 relative to target 220. In particular, by presenting tool 200 to a tracking system with target 220 and target 230 coupled thereto, at least one spatial feature of tool 200 can be identified by target 230, and registered relative to target 220. Subsequently, target 230 is removed from tool 200, replaced with an end effector (e.g. reamer head 210), and tracking of a spatial feature of the end effector is performed by the tracking system relative to target 220. Detailed structures of target 230 are discussed later with reference to FIGS. 3A-3F. Detailed methods for identifying target 230 are discussed later with reference to FIGS. 12 and 13.

As used herein, “spatial feature” refers to a feature of an element which can be used to determine and track aspects of said element. For example, a spatial feature can be a point on a tool, an axis of a tool, or a plane of a tool. Likewise, a spatial feature can also be present with an end effector or a target. Spatial features do not necessarily have to be physically visible features. As examples, spatial features can include: a center of rotation of an end effector; an axis running through a center of a tool, or a plane representing a surface of a target. In this sense, other terms that can at least partially represent a “spatial feature” include: “positional feature”, “directional feature”, “relational feature”, and “locational feature”.

FIG. 2C is an enlarged side view of tool 200 with target 230 coupled to the first end thereof. In the example, target 230 has an optically detectable planar surface 232, and a thickness indicated by distance D₁. In use, a processing unit of a tracking system receives image data from an image sensor of the tracking system, the image data including representations of target 220 and target 230 with which to determine a spatial feature of target 230 based on the image data (in this case, the processing unit determines center P₁ of the optically detectable surface 232 of target 230), and with which to determine a pose of target 220 based on the image data. The processing unit can then determine a spatial relationship between the spatial feature of the target 230 and the pose of the target 220. In this example, a spatial relationship between point P₁ of target 230 and the pose of target 220 is determined.

The processing unit then determines a spatial relationship between target 220 and a spatial feature of the end effector of tool 200 (reamer head 210) based on the spatial relationship between the spatial feature of target 230 (point P₁) and the pose of target 220, and based on a known spatial relationship between the target 230 and the end effector.

In one implementation, for example, the processing unit determines a spatial feature (point P₂) of the tool 200 relative to target 220 based on the spatial relationship between the spatial feature (point P₁) of the target 230 and the pose of the target 220. In the example, point P₂ is a center of the first end of shaft 202. Point P₂ relative to target 220 is determined by modifying coordinates of P₁ relative to target 220 by distance D₁. A spatial feature of the end effector (reamer head 210) is then determined based on a known spatial relationship between the spatial feature of the tool 200 (point P₂) and a spatial feature of the end effector (e.g., a center of rotation of the reamer head 210). For example, the coordinates of point P₂ relative to target 220 can be modified by a known distance. In summary, in this implementation a spatial relationship between a spatial feature of the end effector and the pose of target 220 is determined indirectly, first based on a spatial relationship between target 230 and the tool 200, and a then based on a spatial relationship between tool 200 and the end effector.

In an alternative implementation, a spatial relationship between the spatial feature of the end effector and the pose of target 220 is determined directly, by combining the known spatial relationship between target 230 and tool 200 with the known spatial relationship between tool 200 and the end effector. That is, a single coordinate modification is determined which is a combination of these known physical relationships, and the coordinates of point P₁ relative to target 220 are modified by this single coordinate modification to directly determine the coordinates of the spatial feature of the end effector.

In summary, by determining the spatial relationship between target 220 and target 230, and combining this with known relationships between target 230 and the end effector (reamer head 210), a spatial relationship between the spatial feature of the end effector and the pose of target 220 is determined. This enables the spatial feature of the end effector to be tracked based on target 220 alone even after target 230 is removed. Stated differently, target 220 is indicative of a coordinate frame which moves with tool 200 (a tool coordinate frame), and determining a spatial relationship between the spatial feature of the end effector and the pose of target 220 can be considered as determining a spatial feature of the end effector relative to the tool coordinate frame.

The processing unit can provide the determined spatial relationship between the spatial feature of the end effector and the pose of target 220, for subsequent tracking of the end effector based on target 220. For example, the determined spatial relationship can be provided to a non-transitory processor-readable storage medium of the tracking system, to be accessed before or during tracking of the end effector.

Although FIGS. 2C and 2D illustrate spatial features as points P₁ and P₂, other forms of spatial features are possible. For example, a plane of surface 232 could be identified instead of point P₁. As point P₂, a distance D₁ along an axis which extends normal to the plane of surface 232 could be determined.

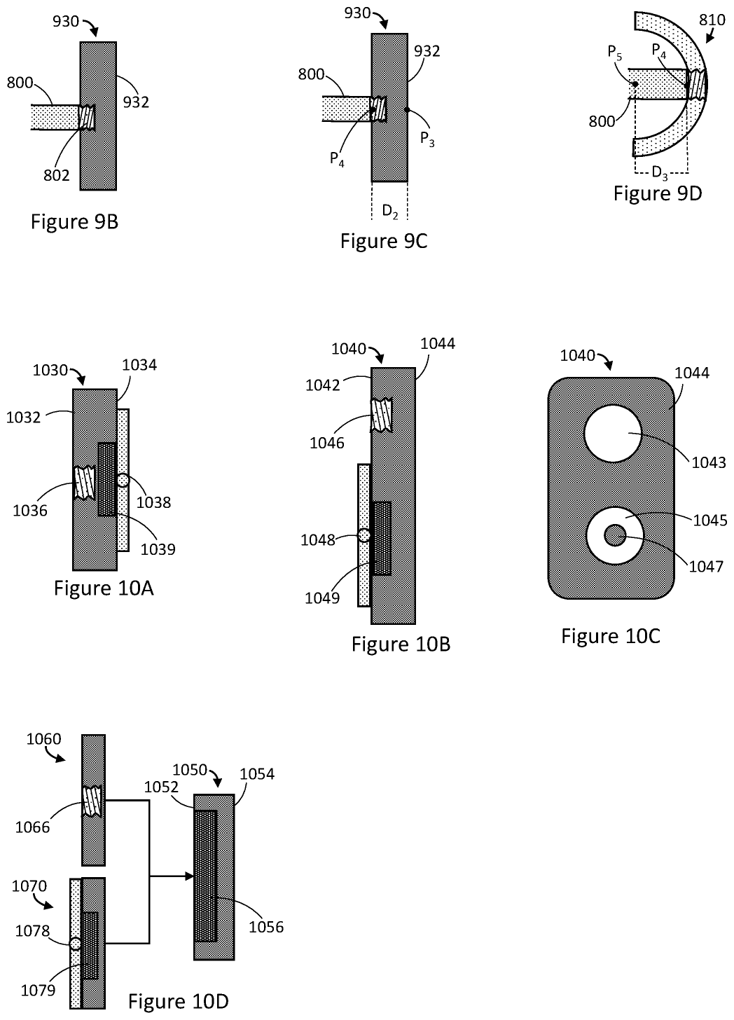

FIG. 2D is an enlarged side view of tool 200, which shows reamer head 210 coupled to the first end of shaft 202. For many reamer heads, the center of rotation of the reamer head aligns with the center of the interface of shaft 202 to which the reamer head is coupled. That is, in many cases the center of rotation of reamer head 210 is point P₂, with no need to account for any offset as mentioned above. Thus, in this example the spatial relationship between the spatial feature of the end effector and the pose of target 220 can be determined by modifying coordinates of point P₁ relative to target 220 by the distance D₁, with no further offsets to account for. This is not necessarily true for all tools, however. In this regard, exemplary cup impactors are discussed later with reference to FIGS. 8A-8C and 9A-9D.

FIG. 2E is a side view of an alternative implementation of a target for determining a spatial feature of the end effector. The example shows a target 240, which can be similar to target 230 discussed above. Discussion of target 230 applies to target 240 unless context dictates otherwise. One difference between target 240 and target 230 is that target 240 is attached with the end effector (reamer head 210) also attached to shaft 202. A spatial feature of the end effector is then determined based on a tracking system analyzing image data of target 220 and target 240, similarly to as discussed above. Another difference between the setup of FIG. 2E and the setup of FIGS. 2B and 2C is that in some implementations of FIG. 2E, target 240 may not be directly analyzed, but instead may act as a contrasting background to reamer head 210, such that a shape of reamer head 210 can be analyzed against this background to determine a spatial feature (e.g. point P₂). In such an implementation, reamer head 210 acts as the “target” as analyzed by the tracking system, whereas target 240 serves a target isolation function to assist with image processing of reamer head 210 as a “target”.

FIGS. 3A, 3B, 3C, 3D, 3E, 3F, 3G, and 3H illustrate exemplary implementations of targets, which could be used as target 230 or target 240 discussed above.

FIG. 3A is a front view of a target 300. Target 300 includes a planar optically detectable surface 302, which is optically detectable by a tracking system. Further, target 300 is shaped as an elliptical disc (in the example circular, though oblong elliptical shapes are also possible). A circular disk shape is rotational symmetrical, which simplifies tracking of the target because there is no need for analysis of orientation of the disk about an axis perpendicular to the planar surface of the disk. However, there are advantages to using a rotationally asymmetric target, as discussed later with reference to FIGS. 3C, 3E, and 3F.

FIG. 3B is an isometric view of the target 300. FIG. 3B shows target 300 as having a cylindrical shape, with a curved side-surface 304, which is adjacent to planar surface 302. Surface 302 is optically detectable relative to surface 304. In an example, surface 302 is made of or coated with a “bright” material, whereas surface 304 is made of or coated with a “dark” material. In this way, when viewed by a tracking system, the shape of surface 302 has high contrast between other elements like a background or side surface 304, such that the shape of surface 302 can be clearly identified.

FIGS. 3C-3H are front views of exemplary targets which can be similar to target 300 in FIGS. 3A and 3B. Description of target 300 is applicable to the targets described with reference to FIG. 3C, 3D, 3E, 3F, 3G, or 3H unless context dictates otherwise.

FIG. 3C shows a target 310, having a rotationally asymmetric optically detectable surface 312. The rotational asymmetry of target 310 allows a tracking system to determine an orientation of target 310 about an axis perpendicular to surface 312 (i.e. orientation about an axis which runs in and out of the page as illustrated). Such orientation can be useful for determining relative rotation between target 220 and target 310 (or any other rotationally asymmetric target).

FIG. 3D shows a target 320, which has a diamond-shaped optically detectable planar surface 322. Such a diamond shape can to some degree indicate orientation about an axis perpendicular to the surface 322, and also illustrates an example where the optically detectable surface in a target need not be elliptical.

FIG. 3E illustrates a target 330a which has a circular shaped surface, which includes a “bright” region 332, and a “dark” region 334. The contrast between region 332 and region 334 creates a rotationally asymmetric pattern on the surface of target 330. As such, even though target 330a has a rotationally symmetric shape (circular), orientation of the target about an axis perpendicular to the surface is still determinable based on the rotationally asymmetric pattern. Although FIG. 3E shows only a single bright region and a single dark region, an appropriate pattern could be designed which includes any appropriate number of bright regions and any number of dark regions. The example of FIG. 3E shows target 330a as having an optically detectable region which extends radially from a center of planar surface 332 to a periphery of planar surface 332, though other shapes of region are possible.

FIG. 3F illustrates a target 330b which has a circular shaped surface, which includes a “bright” region 336, and a “dark” region 338, similarly to target 330a in FIG. 3E. One difference between target 330b and target 330a is that in target 330b, dark region 338 does not extend between a center of the target and a periphery of the target. Instead, dark region 338 is a region which is not centered on the target, and thus creates a rotationally asymmetric pattern on the surface of target 330b. As such, even though target 330b has a rotationally symmetric shape (circular), orientation of the target about an axis perpendicular to the surface is still determinable based on the rotationally asymmetric pattern. Although FIG. 3F shows only a single bright region and a single dark region, an appropriate pattern could be designed which includes any appropriate number of bright regions and any number of dark regions.

FIG. 3G illustrates a target 340 having a circular shaped surface, which includes a “bright” region 342 and a “dark” region 344. A pattern created by contrast between regions 342 and 344 is rotationally symmetric, and thus does not indicate information about orientation of target 340 about an axis perpendicular to a front surface thereof; however, target 340 is still trackable by region 342 alone. That is, FIG. 3G illustrates that it is not necessary for an entire surface of a target to be “bright” or even optically detectable. Although FIG. 3G shows only a single bright region and a single dark region, an appropriate pattern could be designed which includes any appropriate number of bright regions and any number of dark regions.

FIG. 3H illustrates a target 350 having a circular shaped surface, which includes a “bright” region 352 and a “dark” region 354. A pattern created by contrast between regions 352 and 354 is trackable by region 352 alone, but region 354 acts as a contrast region, which prevents environmental light from being misinterpreted by a tracking system as light from the target 350. That is, region 354 acts as a dark region surrounding the bright region 352 so that there is clean delineation between bright region 352 and the environment. Although FIG. 3H shows only a single bright region and a single dark region, an appropriate pattern could be designed which includes any appropriate number of bright regions and any number of dark regions.

FIGS. 4A, 4B, and 4C illustrate an exemplary interface between a tool and an end effector, in this case between a shaft and a reamer head.

FIG. 4A is a rear view of a reamer head 410 not attached to a shaft. In the example, reamer head 410 includes a reamer surface 412 having a plurality of reamer elements 418 (only some labelled in the Figure to avoid clutter), such that when reamer head 410 is positioned in an acetabulum and rotated, the plurality of reamer elements 418 each remove some bone from the acetabulum. Alternative reamer head structures are possible in the context of the present disclosure. Reamer head 410 also includes a pair of cross-bars 414 and 416, which act as one portion of an interface for coupling the reamer head 410 to a shaft. In some implementations, cross-bar 414 can be positioned in a common plane with cross-bar 416. In other implementations, cross-bar 414 and cross-bar 416 can be stacked on top of each other in different planes. In yet other implementations, a single cross-bar could be used. Cross-bars 414 and 416 are illustrated as being perpendicular to each other, but this is not necessarily the case; in some implementations cross-bars 414 and 416 can be non-parallel to each other, but also non-perpendicular to each other.

FIG. 4B is a side cross-sectional view of reamer head 410 and a shaft 400 to which reamer head 410 is to be coupled. Shaft 400 has a plurality of slots 402 for receiving portions of cross-bar 414 or and/or cross-bar 416, and a plurality of protrusions 404 for retaining the portions of cross-bar 414 or and/or cross-bar 416 in respective slots 402. Slots 402 and protrusions 404 act as another interface portion, which cooperates with the interface portion of the reamer head 410 (cross bars 414 and 416) to couple shaft 400 to reamer head 410. Although FIG. 4B illustrates an exemplary slot 402 and protrusion 404 which hold a portion of cross-bar 416 during clockwise rotation of reamer head 410, other mechanisms are possible, such as clips, screws, magnets, or other fasteners or combinations of fasteners.

FIG. 4C is a side cross-sectional view which shows shaft 400 coupled to reamer head 410. As mentioned above, one interface portion (cross-bars 414 and 416) cooperates with another interface portion (slots 402 and protrusions 404) to couple shaft 400 to reamer head 410. The illustrated interface portions are a removable coupling.

FIGS. 5A and 5B are side cross-sectional views which illustrate an exemplary coupling between a shaft and a target. FIG. 5A is a side view of a target 530 and shaft 400 to which target 530 is to be coupled. Target 530 can be used for example as target 230 discussed above with reference to FIG. 2B and 2C, and target 530 could have any of the shapes or features discussed with regard to targets 300, 310, 320, 330a, 330b, 340 or 350 discussed above with reference to FIGS. 3A-3G. Discussion of targets 230, 300, 310, 320, 330, 330a, 330b, 340 and 350 are applicable to target 530 unless context dictates otherwise. Target 530 includes an optically detectable surface 532, for detection by a tracking system. The description of shaft 400 above with reference to FIGS. 4B and 4C is also applicable to FIGS. 5A and 5B. In this example, shaft 400 in FIGS. 4B and 4C is the same shaft as shaft 400 in FIGS. 5A and 5B, with reamer head 410 and target 530 being interchangeably couplable to shaft 400. To this end, target 530 comprises an interface portion including cross-bars 534 and 536, which can be similar to cross-bars 414 and 416. Similar to the cross-bars discussed with reference to FIG. 4A, in some implementations, cross-bar 534 can be positioned in a common plane with cross-bar 536; in other implementations, cross-bar 534 and cross-bar 536 can be stacked on top of each other in different planes; in yet other implementations, a single cross-bar could be used. In some implementations, cross-bars 534 and 536 are integrated with target 530. In other implementations, cross-bars 534 and 536 are separate components coupled to target 530. Cross-bars 534 and 536 are illustrated as being perpendicular to each other, but this is not necessarily the case; in some implementations cross-bars 534 and 536 can be non-parallel to each other, but also non-perpendicular to each other.

FIG. 5B is a side-view which illustrates shaft 400 coupled to target 530, in a similar manner to as described with reference to FIGS. 4B and 4C. One interface portion (cross-bars 534 and 536) cooperates with another interface portion (slots 402 and protrusions 404) to couple shaft 400 to target 530. The illustrated interfaces are a removable coupling.

Target 530 illustrated in FIGS. 5A and 5B includes a recess 538 to accommodate shaft 400 when target 530 is coupled thereto. However, depending on the nature of coupling between shaft 400 and target 530, such as recess is not strictly necessary.

To register the tool discussed with reference to FIGS. 4A-4C and 5A-5B, target 530 is attached to shaft 400, with another target (such as target 220 discussed above with reference to FIGS. 2A, 2B, and 2E) attached to another portion of the tool. A tracking system captures or receives image data including representations of the two targets, and determines a spatial relationship between a spatial feature of the tool or end effector (reamer head 410) and a pose of target 220, based on the positions of the two targets in the image data. This process is described in detail above and below with reference to FIGS. 2C, 9C, 9D, and 12. The target 530 is then removed from shaft 400, and the reamer head 410 is attached to shaft 400. The tool is thus registered for use with tracking of only target 220.

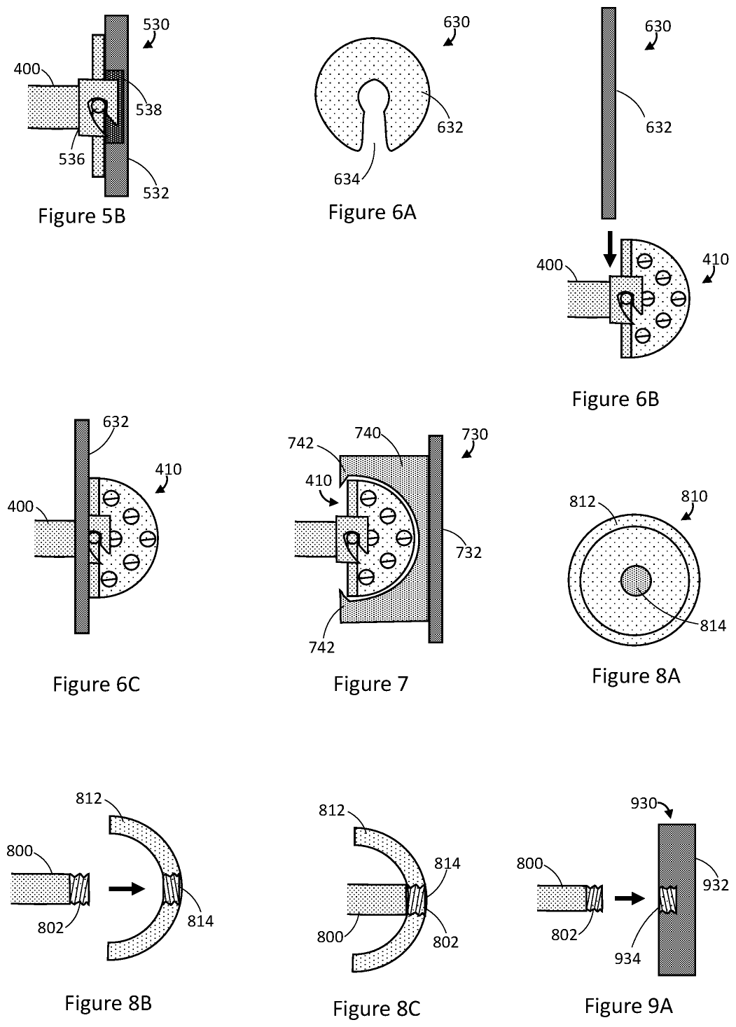

FIGS. 4A-4C and 5A-5B illustrate an exemplary interface for coupling end effectors and targets to a tool. However, other interfaces are possible, as discussed below with reference to FIGS. 6A-6C, 7, 8A-8C, 9A-9D, and 10.

FIGS. 6A, 6B, 6C, and 7 illustrate exemplary interfaces for coupling a target to a tool/end effector with the end effector in place.

FIG. 6A is a front view of an exemplary target 630, which can for example be used as target 240 in FIG. 2E discussed above, and can include similar features to targets 300, 310, 320, 330, 340 or 350 discussed above with reference to FIGS. 3A-3g. Discussion of targets 240, 300, 310, 320, 330a, 330b, 340, and 350 is applicable to target 630 unless context dictates otherwise. Target 630 includes an optically detectable surface 632 for detection by a tracking system. Target 630 also includes a slot or recess 634, which acts as one interface portion for coupling to a tool or end effector.

FIG. 6B is a side cross-sectional view of target 630 to be coupled to shaft 400 and reamer head 410, which are as described above with reference to FIGS. 4A-4C. Target 630 is slid onto shaft 400, with shaft 400 being received in recess 634. In this way, target 630 is positioned on the tool behind reamer head 410.

FIG. 6C is a side cross-sectional view of target 630 positioned behind reamer head 410, coupled to the tool. Target 630 can be retained on shaft 400 by frictional force, non-permanent adhesive, clips, alignment features on the shaft or target, or any other appropriate fastener or combinations of fasteners. One interface portion (recess 634) cooperates with another interface portion (a shape of shaft 400) to couple shaft 400 to target 630.

In use, target 630 is coupled to the tool, along with another target (such as target 220 discussed above with reference to FIGS. 2A, 2B, and 2E) and a tracking system captures or receives image data including representations of the two targets, and determines a spatial relationship between a spatial feature of the tool or end effector (reamer head 410) and the target 220, based on the positions of the two targets in the image data. The target 630 is then removed from shaft 400. The tool is thus registered for use with tracking of only target 220. Similar to as discussed above regarding FIG. 2E, in some implementations, target 630 may not be directly analyzed, but instead may act as a contrasting background to reamer head 410, such that a shape of reamer head 410 can be analyzed against this background to determine a spatial feature and register the tool. In such an implementation, reamer head 410 acts as the “target” as analyzed by the tracking system, whereas target 630 serves a target isolation function to assist with image processing of reamer head 410 as a “target”.

FIG. 7 is a side cross-sectional view of another exemplary interface for coupling a target to a tool. FIG. 7 shows an exemplary target 730, which can be similar to targets 230, 240, 300, 310, 320, 330a, 330b, 340 and 350 discussed with reference to FIGS. 2B, 2C, 2E, 3A, 3B, 3C, 3D, 3E, 3F, 3G, and 3H above. Discussion of targets 230, 240, 300, 310, 320, 330a, 330b, 340, and 350 is applicable to target 730 unless context dictates otherwise. Target 730 includes an optically detectable surface 732 for detection by a tracking system. Target 730 is coupled to or integral with an adapter 740, which is removably couplable to an end effector to the tool. In the illustrated example, adapter 740 fits around and clips to reamer head 410 as coupled to shaft 400. Clips 742 of adapter 740 retain adapter 740 on reamer head 410.

In use, target 730 is coupled to the tool, along with another target (such as target 220 discussed above with reference to FIGS. 2A, 2B, and 2E) and a tracking system captures or receives image data including representations of the two targets, and determines a spatial relationship between a spatial feature of the tool or end effector (reamer head 410) and the target 220, based on the positions of the two targets in the image data. Adapter 740 and target 730 are then removed from the tool. The tool is thus registered for use with tracking of only target 220.

FIGS. 4A-4C, 5A-5B, 6A-6C, and 7 illustrate calibration of a reamer as an example tool. However, the present disclosure applies to other tools as well.

FIGS. 8A, 8B, and 8C illustrate an exemplary interface between a tool and an end effector, in this case between a shaft of a cup impactor and a cup.

FIG. 8A is a rear view of a cup 810 not attached to a shaft. In the example, cup 810 includes a cup body 812. Cup 810 also includes a threaded recess 814, which acts as one portion of an interface for coupling the cup 810 to a shaft.

FIG. 8B is a side cross-sectional view of cup 810 and a shaft 800 to which cup 810 is to be attached. Shaft 800 has a threaded portion 802, to be received in threaded recess 814 of cup 810. In this way, threaded portion 802 acts as one interface portion, which cooperates with another interface portion of cup 810 (threaded recess 814) to couple shaft 800 to cup 810.

FIG. 8C is a side cross-sectional view which shows shaft 800 coupled to cup 810. The illustrated interface portions are a removable coupling. Although FIGS. 8A-8C illustrate an exemplary threaded portion and cooperating recess, other mechanisms are possible, such as clips, screws, magnets, or other fasteners or combinations of fasteners.

FIGS. 9A and 9B are side cross-sectional views which illustrate an exemplary coupling between a shaft and a target. FIG. 9A is a side view of a target 930 and shaft 800 to which target 930 is to be attached. Target 930 can be used as, for example, target 230 discussed above with reference to FIG. 2B and 2C, and target 930 could have any of the shapes or features discussed regarding targets 300, 310, 320, 330a, 330b, 340 or 350 discussed above with reference to FIGS. 3A-3H. Discussion of targets 230, 300, 310, 320, 330a, 330b, 340, and 350 is applicable to target 930 unless context dictates otherwise. Target 930 includes an optically detectable surface 932, for detection by a tracking system. The description of shaft 800 above with reference to FIGS. 8B and 8C is also applicable to FIGS. 9A and 9B. In this example, shaft 800 in FIGS. 8B and 8C is the same shaft as shaft 800 in FIGS. 8A and 8B, with cup 810 and target 930 being interchangeably couplable to shaft 800. To this end, target 930 comprises an interface portion including threaded recess 934, which can be similar to threaded recess 814.

FIG. 9B is a side cross-sectional view which illustrates shaft 800 coupled to target 930, in a similar manner to as described with reference to FIGS. 8B and 8C. One interface portion (threaded portion 802) cooperates with another interface portion (threaded recess 934) to couple shaft 800 to target 930. The illustrated interface portions are a removable coupling.

To register the tool discussed with reference to FIGS. 8A-8C and 9A-9B, target 930 is attached to shaft 800, with another target (such as target 220 discussed above with reference to FIGS. 2A, 2B, and 2E) attached to another portion of the tool. A tracking system captures or receives image data including representations of the two targets, and determines a spatial relationship between a spatial feature of the tool or end effector (cup 810) and a pose of the target 220 based on the positions of the two targets in the image data. The target 930 is then removed from shaft 800, and the cup 810 is attached to shaft 800. The tool is thus registered for use with tracking of only target 220.

FIG. 9C is a side cross-sectional view of shaft 800 with target 930 coupled to the first end thereof. FIG. 9C is similar to FIG. 9B, except that different features are labelled to avoid excessive clutter within a single Figure. In the example, target 930 has an optically detectable planar surface 932, and a thickness indicated by distance D₂. In use, a processing unit of a tracking system receives image data from an image sensor of the tracking system, the image data including representations of target 930 and another target (such as target 220 discussed with reference to FIGS. 2A, 2B and 2E), with which to determine a spatial feature of target 930, and with which to determine a pose of the other target (target 220). In this example, the processing unit determines a position of center P₃ of the optically detectable surface 932 of target 930. The processing unit also determines a pose of the other target (target 220) based on the image data, and determines a spatial relationship between the spatial feature of the target 930 and the pose of the target 220 based on the image data. In this example, the spatial relationship between point P₃ of target 930 and the pose of target 220 is determined.

The processing unit can then determine a spatial relationship between a spatial feature of the end effector to be attached to shaft 800 (cup 810) based on the spatial relationship between the spatial feature of target 930 and the pose of target 220, and based on a known spatial relationship between the target 930 and the end effector.

In one implementation, the processing unit determines a spatial relationship between a spatial feature (point P₄) of the shaft 800 and the pose of target 220, based on the spatial relationship between the spatial feature (point P₃) of the target 930 and the pose of the target 220. In the example, point P₄ is at the center of the interface of the first end of shaft 800. The spatial relationship between point P₄ and the pose of target 220 is determined by modifying coordinates of P₃ relative to target 220 by distance D₂. A spatial relationship between a spatial feature of the end effector (cup 810) and the pose of target 220 is then determined based on a known spatial relationship between the spatial feature of the shaft (point P₄) and a spatial feature of the end effector (e.g., a center of rotation of the cup 810). FIG. 9D illustrates an example.

FIG. 9D is a side cross-sectional view of shaft 800 coupled to cup 810. A center of rotation of cup 810 (a spatial feature) is indicated as point P₅, and is separated from point P₄ by a distance D₃. To determine a spatial relationship between the spatial feature of the end effector and the pose of target 220, the coordinates of point P₄ relative to target 220 can be modified by distance D₃. To summarize this implementation, a spatial relationship between the spatial feature of the end effector and the pose of target 220 is determined indirectly in steps, based on a physical relationship between target 930 and shaft 800, and subsequently based on a physical relationship between shaft 800 and the end effector (cup 810).

In an alternative implementation, a spatial relationship between the spatial feature of the end effector and the pose of target 220 is determined directly, by combining the known spatial relationship between target 930 and shaft 800 with the known spatial relationship between shaft 800 and the end effector. That is, a single modification is determined which is the sum of the known physical relationships (i.e. the sum of D₂ and D₃), and the coordinates of point P₃ relative to target 220 are modified by the single modification to directly determine the coordinates of the spatial feature of the end effector relative to target 220.

In summary, by determining the spatial relationship between target 220 and target 930, and combining this with known relationships between target 930, shaft 800, and the end effector, a spatial relationship between a spatial feature of the end effector and the pose of target 220 is determined. This enables the spatial feature of the end effector to be tracked based on target 220 alone even after target 930 is removed. Stated differently, target 220 is indicative of a coordinate frame which moves with the cup impactor (a tool coordinate frame), and determining a spatial relationship between the spatial feature of the end effector and the pose of target 220 can be considered as determining a spatial feature of the end effector relative to the tool coordinate frame.

The processing unit can provide the determined spatial relationship between the spatial feature of the end effector and the pose of target 220, for subsequent tracking of the end effector based on target 220. For example, the determined spatial relationship can be provided to a non-transitory processor-readable storage medium of the tracking system, to be accessed before or during tracking of the end effector.

Although FIGS. 9C and 9D illustrate spatial features as points P₃, P₄, and P₅, other forms of spatial features are possible. For example, a plane of surface 932 could be identified instead of point P₃. As point P₄, a distance D₂ along an axis which extends normal to the plane of surface 932 could be determined. Instead of point P₅, a plane on a rear surface of the cup could be determined.

The targets described herein can be couplable to more than one tool. For example, multiple different tools may have the same interface portion, such that a cooperating interface portion on a target can couple with each of the tools. As another example, a single target may include multiple interface portions, to couple to different interface portions of different tools. Examples are discussed below with reference to FIGS. 10A-10D.

FIG. 10A is a side cross-sectional view of a target 1030, which can be similar to any of targets 230, 530, 630, 730, or 930 discussed herein. Discussion of targets 230, 530, 630, 730, and 930 is applicable to target 1030 unless context dictates otherwise. Target 1030 has a first optically detectable surface 1032, and a second optically detectable surface 1034 which is opposite surface 1032. Target 1030 also has one interface portion 1036, illustrated as a threaded recess similar to threaded recesses 814 and 834 discussed above. Target 1030 also has another interface portion 1038, illustrated as cross-bars similar to cross bars 414, 416, 534, and 536 discussed above. Interface portions 1036 and 1038 are positioned on opposite sides of target 1030. Target 1030 is also illustrated as having a recess 1039 for accommodating a cooperating interface portion which couples to interface portion 1038. Recess 1039 is optional depending on the structure of interface portion 1038 and the cooperating interface portion.

In one use case, interface portion 1036 couples to a cooperating interface portion on a tool, such as threaded portion 802 on shaft 800 of a cup impactor. Optically detectable surface 1034 can then be viewed by a tracking system for registration of the tool similarly to as discussed above. In another use case, interface portion 1038 couples to a cooperating interface portion on a tool, such as recess 402 and protrusion 404 on shaft 400 of a reamer. Optically detectable surface 1032 can then be viewed by a tracking system for registration of the tool similarly to as discussed above. As discussed above with reference to FIG. 3G, it is not necessary for an entire planar surface of the target to be optically detectable, so the presence of interface portion 1036 on surface 1032, and the presence of interface portion 1038 on surface 1034 does not prevent optical detection of the planar surfaces.

In the example of FIG. 10A, different interface portions to which different tools are coupled are on opposite sides of the target. In other implementations however, this is not necessarily the case. For example FIG. 10B is a side cross-sectional view of a target 1040, in which a plurality of different interface portions are positioned on a common side of the target, with an opposing side comprising an optically detectable surface used for tracking when the target is used with different tools. Specifically target 1040 has a first surface 1042, with interface portion 1046 and 1048 being positioned thereon. Interface portion 1046 is illustrated as a threaded recess similar to threaded recesses 814 and 834 discussed above. Interface portion 1048 is illustrated as cross-bars similar to cross bars 414, 416, 534, and 536 discussed above. Target 1040 is also illustrated as having a recess 1049 in surface 1042 for accommodating a cooperating interface portion which couples to interface portion 1048. Recess 1049 is optional depending on the structure of interface portion 1048 and the cooperating interface portion. The form of interface portions 1046 and 1048 is merely exemplary, and alternative interface portions could be implemented having different form or shape. Further, additional interface portions could be added as appropriate.

Target 1040 has a second surface 1044 opposite surface 1042, with surface 1044 comprising at least one optically detectable region. FIG. 10C is a front view of target 1040, showing the surface 1044. Target 1040 comprises an optically detectable region 1043 and an optically detectable region 1045 on surface 1044, with optically detectable regions 1043 and 1045 contrasting with other regions of surface 1044. In some implementations, a single optically detectable region or pattern could be provided on surface 1044, but if this optically detectable region is offset from an axis of a tool being registered, registration can be complicated. In the illustrated implementation, optically detectable region 1043 is analyzed by a tracking system when registering a first tool, and optically detectable region 1045 is analyzed by the tracking system when registering a second tool. In the example, when a cup impactor shaft is coupled to interface portion 1046, a tracking system analyzes optically detectable region 1043 to register the cup impactor. When a reamer shaft is coupled to interface portion 1048, a tracking system analyzes optically detectable region 1045 to register the reamer. This arrangement having an optically detectable region corresponding to, aligned with, or opposite to each interface portion enables each optically detectable portion to be aligned with an axis of the tool which couples to the corresponding interface portion, such that registration is simplified. Surface 1044 is also shown as having a “dark” region 1047, which contrasts with region 1045; this acts as a pattern by which a tracking system can identify optically detectable regions 1043 and 1045, and analyze the correct region based on what tool is being registered. In the example, when registering a cup impactor, the tracking system analyzes the solid elliptical region, whereas when registering a reamer, the tracking system analyzes the donut shaped region. Different patterns could be used as appropriate.

FIG. 10D is a side cross-sectional view of a target 1050 which uses an adapter to accommodate multiple interface portions for coupling with different tools. In particular, target 1050 has a surface 1052 having an adapter coupler 1056 (illustrated as a recess) therein for receiving one of a plurality of candidate adapters. Target 1050 has an optically detectable surface 1054 opposite surface 1052. FIG. 10D also shows two exemplary adapters 1060 and 1070, though additional adapters could also be used. One of adapters 1060 or 1070 is inserted into adapter coupler 1056, and coupled there by any appropriate means, such as friction, clips, magnets, screws, pins, non-permanent adhesive, or any other fastener or combination of fasteners. Adapter 1060 has a threaded recess 1066 therein for coupling to a cup impactor shaft, and adapter 1070 has a cross-bar interface portion 1078 for coupling to a reamer shaft. Adapter 1070 is also illustrated as having a recess 1079 for accommodating a cooperating interface portion which couples to interface portion 1078. Recess 1079 is optional depending on the structure of interface portion 1078 and the cooperating interface portion. Adapters can be coupled first to a corresponding tool then to target 1050, or adapters can be coupled first to target 1050 then coupled to a corresponding tool. Using adapters as in FIG. 10D advantageously can provide a vast number of tool interface portions for coupling, with requiring a vast number of targets.

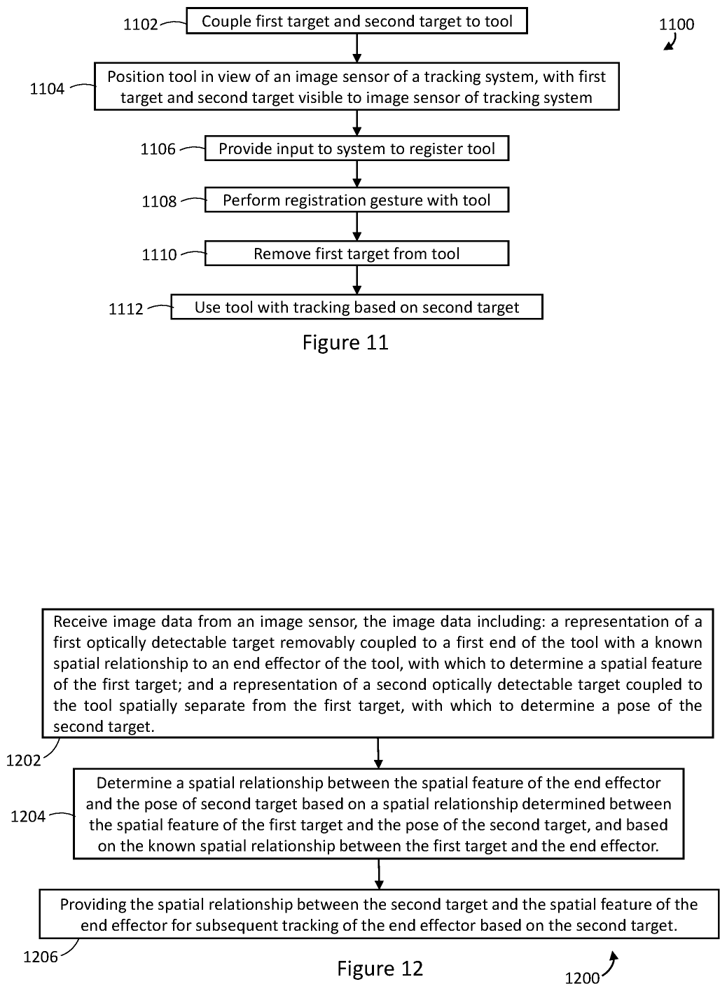

FIG. 11 is a flowchart diagram which illustrates a method 1100 for registering a tool, using the targets and features described above. Method 1100 can be performed by an operator of a tool or an assistant to said operator. Method 1100 is shown as including acts 1102, 1104, 1106, 1108, 1110 and 1112; however additional acts could be added, or acts could be removed or rearranged, as appropriate for a given application.

In act 1102, a first target and a second target are coupled to a tool. Such a first target could be for example any of targets 230, 240, 300, 310, 320, 330a, 330b, 340, 350, 530, 630, 730, 930, 1030, 1040, or 1050 discussed above. Such a second target could be for example target 220 discussed with reference to FIG. 2A, 2B, or 2E.

In act 1104, the tool is positioned in view of an image sensor of a tracking system, with the first and second targets visible to the tracking system.

In act 1106, input is provided to the tracking system to cause the system to register the tool. For example, an operator could press a button instructing the tracking system to begin to capture image data for registration. In some implementations, act 1104 can be performed after act 1106. For example, an operator may instruct the tracking system to begin capturing image data, then move the tool in view of the image sensor. In response to the instruction to register the tool, the tracking system can register the tool using any of the hardware or techniques described herein, such as described in method 1200 discussed below with reference to FIG. 12.

In act 1108, a registration gesture is performed. This act is optional, but may improve accuracy of the registration. As an example, the operator could wave the tool in front of the image sensor, or otherwise move the tool through some motion, during which the tracking system captures multiple images of multiple views of the tool and targets. These multiple images when analyzed can provide a more diverse representation of the tool and thus more accurate analysis.

In act 1110, the first target is removed from the tool, leaving only the second target coupled to the tool.

In act 1112, the tool is used in accordance with its intended function, and is tracked by the tracking system based only on tracking of the second target.

In summary, features of the tool are registered to the second target based on the first target, such that the features can be subsequently tracked based only on the second target.

FIG. 12 is a flowchart diagram which illustrates a computer implemented method 1200 for registering a tool using any of the hardware or techniques described herein. Method 1200 can be performed by a tracking system, such as that described with reference to FIG. 1. In particular, processing acts are performed by a processing unit of such a tracking system, such as a processing unit in computing device 132. Method 1200 is illustrated as including acts 1202, 1204, and 1206. However, additional acts could be added, or acts could be removed or rearranged, as appropriate for a given application.

In act 1202, image data is received from an image sensor of the tracking system, for determination of a spatial feature of a first target and a pose of a second target. The image data includes a representation of the first target (for example any of targets 230, 240, 300, 310, 320, 330a, 330b, 340, 350, 530, 630, 730, 930, 1030, 1040, or 1050 discussed above) removably coupled to a first end of the tool with a known spatial relationship to an end effector of the tool. The first target is optically detectable to the image sensor. The image data also includes a representation of a second target (such as target 220 discussed with reference to FIG. 2A, 2B, or 2E above) coupled to the tool spatially separate from the first target. The second target is optically detectable to the image sensor.

A spatial feature of the first target is determined based on the image data. In some implementations, pose of the first target in six degrees of freedom can be determined, such as when rotationally asymmetric targets or patterns are used (for example as in targets 310, 330a, and 330b in FIGS. 3C, 3E, and 3F). In other implementations, only certain features of the target could be determined in less degrees of freedom. For example, when a rotationally symmetric target (for example targets 300, 340, or 350 in FIGS. 3A, 3G, and 3H), certain aspects of the first target's pose, namely rotation about an axis perpendicular to an optically detectable surface of the target, may not be determined. An exemplary technique for determining pose or spatial features of the first target is discussed later with reference to FIG. 13. Examples of features which can be determined are described above with reference to FIGS. 2C and 9C.

A pose of the second target is determined based on the image data. Preferably the pose of the second target is determined in six degrees of freedom, for optimum accuracy in tracking of the tool later. Determination of pose of the second target can be performed for example as discussed with reference to target 114 in FIG. 1.

In act 1204, a spatial relationship between a spatial feature of an end effector of the tool and the pose of the second target is determined based on a spatial relationship determined between the spatial feature of the first target and the pose of the second target, and based on a known spatial relationship between the first target and the end effector. The determined spatial relationship between the spatial feature of the first target and the pose of the second target can be, for example, a vector from a point fixed relative to the second target, to a point fixed relative to the first target. This determination can be made, for example, by comparing coordinates of the spatial feature of the first target to coordinates of the second target. Other relationships are possible, including between points, axes, and planes. The discussion above regarding FIGS. 2C, 9C, and 9D describe this process in detail. For example, as discussed above with reference to FIG. 2C, a point P₁ on the first target may be offset from a point P₂ on the tool by a known distance D₁. Further the point P₂ on the tool may be offset from a spatial feature of the end effector by another known distance. Many reamers are designed with the interface between the shaft and the reamer head being the center of rotation of the reamer head (which is also a desired spatial feature to be determined and tracked of the reamer head). Thus, in the case of reamers this another known distance may be 0. However, this is not necessarily true for all tools. For example, as discussed above with reference to FIGS. 9C and 9D, in the case of cup impactors, the interface between the shaft and cup is often positioned at a non-zero distance from the center of rotation of the cup, such that the another known distance (i.e. D₃ in FIG. 9D) is non-zero.

The known distances can be combined to provide a single offset to directly determine a spatial relationship between the first target and the end effector, which in turn can be used to determine a spatial relationship between the spatial feature of the end effector and the pose of the second target in act 1208. Alternatively, the known distances can be applied separately to perform act 1210 indirectly. In particular, a spatial relationship between the pose of the second target and the spatial feature on the tool (e.g. point P₂ in FIG. 2C, or point P₄ in FIGS. 9C and 9D) can first be determined. Subsequently, a spatial relationship between the pose of the second target and a spatial feature of the end effector can be determined based on the determined spatial relationship between the pose of the second target and the spatial feature on the tool, and a known spatial relationship between the point on the tool (point P₂ or point P₄) and the spatial feature of interest on the end effector (e.g. point P₅ in FIG. 9D).

In act 1206, the determined spatial relationship between the second target and the spatial feature of the end effector is provided, for subsequent tracking of the end effector based on the second target. For example, the determined spatial relationship between the second target and the spatial feature of the end effector can be stored in a non-transitory processor-readable medium of a tracking system, for subsequent retrieval or access by the tracking system. As needed before or during a tracking operation, the stored spatial relationship is accessed, and applied by a tracking system to track the end effector of the tool relative to the second target.

Additionally, a non-transitory processor-readable medium of a tracking system may store a model of the first target, which is accessed and used to determine the spatial feature of the first target based on the image data. A non-transitory processor-readable medium of a tracking system may store a model of the second target, which is accessed and used to determine the pose of the second target based on the image data.

FIG. 13 illustrates an exemplary technique for determining a spatial feature or pose of a target. FIG. 13 illustrates a ray model for fitting a target shape. The target in this example has a circular shape, though other shapes are possible. A periphery of a target as represented in image data is first identified (or more particularly, a periphery of an optically detectable region of the target is identified, for targets where the physical periphery is not the same as the periphery of the optically detectable region, such as target 350 in FIG. 3H). Then, a plurality of rays are modeled extending from the image sensor to the periphery of the disk. FIG. 13 shows a first set of rays 1302 extending from an image sensor in a first position, and a second set of rays 1304 extending from an image sensor in a second position. Such different positions of image sensor could be achieved by using a tracking system with multiple image sensors in different positions, and simultaneously capturing image data with each image sensor. Alternatively, in tracking systems with a single image sensor, either the tool or the image sensor could be moved (such as through a registration gesture) to obtain image data of the tool and targets from different angles. FIG. 13 only shows two sets of rays representing two image sensor positions, but in practice many more sets of rays could be modelled, representing many more image sensor positions. Modelling multiple sets of rays from different views improves accuracy of the pose determination. However, in some implementations it may be possible to achieve sufficient accuracy with only a single image of image data from one viewpoint. Thus, multiple images in the image data are not necessarily required.

A model of the target is then fitted to the plurality of rays. The model is provided to the tracking system, and includes at least an expected shape of the optically detectable portion of the target. In the illustrated example, the tracking system can know that the target has a circular shape. In some implementations, the tracking system may not be aware of the diameter of the circle or other spatial features of the target. In other implementations, the tracking system may know the exact dimensions of the target. Even if the exact dimensions are known however, the tracking system may be flexible with fitting to such dimensions, to accommodate issues such as image bloom or other artifacts. The fit could for example be a fit that minimizes standard deviation between the modelled target and the plurality of rays. In the example, a target with a circular shape is used, and a model 1306 of the circular shape is orientated in 3D space so as to align with the model of the plurality of rays. The position of the model of the target which best matches the ray model is indicative of the pose of the target.

Although FIG. 13 only shows the model of one target (the ellipse), the second target should also be represented in the image data, and a pose determined thereof. In this way the determined pose of the first target can be correlated to the second target to determine a spatial relationship therebetween.

With reference again to FIG. 1, a system for registering a tool is described, in which any of the targets and tools described herein can be used. The system includes an image sensor (image sensor 122 in FIG. 1), and a processing unit (such as a processor in computing device 132 in FIG. 1). In use, the image sensor captures image data including representations of a first target (e.g. target 230, 240, 300, 310, 320, 330, 340, 530, 630, 730, 930, or 1030) and a second target (e.g. target 220). Such image data can be a still image, or multiple images, such as a video stream. The captured image data is directed from image sensor 122 to the processing unit (for example by a direct connection or over a network). The processing unit receives the image data, and determines a spatial relationship between a spatial feature of an end effector and a pose of the second target based on a spatial relationship between the first target and the second target and a known spatial relationship between the first target and the end effector, such as according to method 1200 in FIG. 12.

In some implementations, the tracking system includes a non-transitory processor-readable storage medium which stores instructions thereon. When executed, said instructions cause the processing unit to perform the actions described above. In other implementations, the processing unit comprises a logic circuit or similar which can perform processing operations without needing to read instructions from a medium.

The various computing devices shown herein can comprise a processing unit (for example a microprocessor, FPGA, ASIC, logic controller, or any other appropriate processing hardware), a storage device (e.g. non-transitory processor-readable storage medium, such as memory, RAM, ROM, magnetic-disk, solid state storage, or any other appropriate storage hardware) storing instructions which when and executed by the processing unit configure the computing device to perform operations for example to provide the functionality and features described herein. Computer program code for carrying out operations may be written in any combination of one or more programming languages, e.g., an object oriented programming language such as Java, Smalltalk, C++ or the like, or a conventional procedural programming language, such as the “C” programming language or similar programming languages.

Any of the computing devices may have communication subsystems to communicate via a network. Any may have a display device and other input and/or output devices.

Practical implementation may include any or all of the features described herein. These and other aspects, features and various combinations may be expressed as methods, apparatus, systems, means for performing functions, program products, and in other ways, combining the features described herein. A number of embodiments have been described. Nevertheless, it will be understood that various modifications can be made without departing from the spirit and scope of the processes and techniques described herein. In addition, other steps can be provided, or steps can be eliminated, from the described process, and other components can be added to, or removed from, the described systems. Accordingly, other embodiments are within the scope of the following claims.

Throughout the description and claims of this specification, the word “comprise”, “contain” and variations of them mean “including but not limited to” and they are not intended to (and do not) exclude other components, integers or steps. Throughout this specification, the singular encompasses the plural unless the context requires otherwise. In particular, where the indefinite article is used, the specification is to be understood as contemplating plurality as well as singularity, unless the context requires otherwise.

Features, integers, characteristics, or groups described in conjunction with a particular aspect, embodiment or example of the invention are to be understood to be applicable to any other aspect, embodiment or example unless incompatible therewith. All of the features disclosed herein (including any accompanying claims, abstract and drawings), and/or all of the steps of any method or process so disclosed, may be combined in any combination, except combinations where at least some of such features and/or steps are mutually exclusive. The invention is not restricted to the details of any foregoing examples or embodiments. The invention extends to any novel one, or any novel combination, of the features disclosed in this specification (including any accompanying claims, abstract and drawings) or to any novel one, or any novel combination, of the steps of any method or process disclosed.

1. A system for registering a tool for tracking of said tool, the tool comprising a first end for use with an end effector, the system comprising:
an image sensor;
a first target removably couplable to the first end of the tool with a known spatial relationship to the end effector, the first target being optically detectable to the image sensor;
a second target couplable to the tool spatially separate from the first target, the second target being optically detectable to the image sensor; and
a processing unit configured to:
receive image data from the image sensor, the image data including representations of the first target and the second target with which to determine a spatial feature of the first target and determine a pose of the second target;
determine a spatial relationship between the second target and a spatial feature of the end effector, based on a spatial relationship determined between the spatial feature of the first target and the pose of the second target, and based on the known spatial relationship between the first target and the end effector; and
provide the spatial relationship between the second target and the spatial feature of the end effector, for subsequent tracking of the end effector based on the second target.

2. The system of claim 1, wherein the first target comprises an optically detectable planar surface.

3. The system of claim 1, wherein the first target comprises an optically detectable planar disk.

4.-5. (canceled)

6. The system of claim 1, wherein the second target comprises a plurality of optically detectable markers coupled to a support, the support removably couplable to the tool.

7. The system of claim 1, wherein the first target is removably couplable to the tool concurrently with the end effector coupled to the first end of the tool.

8. The system of claim 7, further comprising an interface to removably couple the first target to the end effector.

9. (canceled)

10. The system of claim 1, wherein the end effector is removably couplable to the first end of the tool by an interface, and the first target is removably couplable to the first end of the tool by the interface with the end effector absent.

11. The system of claim 1, wherein:
the tool is a reamer comprising a shaft and a reamer head, the reamer head being the end effector and comprising a first interface portion, the shaft comprising a second interface portion removably couplable to the first interface portion of the reamer head;
the first target comprises a third interface portion removably couplable to the second interface portion of the shaft; and
the known spatial relationship between the first target and the end effector comprises a known offset between a center of the first target when removably coupled to the shaft and a center of rotation of the reamer head.

12. The system of claim 1, wherein:
the tool is a cup impactor comprising a shaft and a cup, the cup being the end effector and comprising a first interface portion, the shaft comprising a second interface portion removably couplable to the first interface portion of the cup;
the first target comprises a third interface portion removably couplable to the second interface portion of the shaft; and
the known spatial relationship between the first target and the end effector comprises a known offset between a center of the first target when removably coupled to the shaft and a center of rotation of the cup.

13. The system of claim 1, wherein the tool is one of a plurality of candidate tools, the first target comprising a plurality of interface portions, each interface portion configured to removably couple to a cooperating interface portion on at least one tool of the plurality of candidate tools.

14. (canceled)

15. The system of claim 13, wherein the plurality of interface portions comprise at least a first interface portion and a second interface portion, the first interface portion and the second interface portion positioned on a first side of the first target, further wherein at least one optically detectable region is positioned on a second side of the first target opposite the first side.

16.-18. (canceled)

19. The system of claim 1, wherein the first target comprises a circular optically detectable region, and wherein to determine a spatial feature of the first target comprises:
identifying a periphery of the optically detectable region as represented in the image data;
modelling a plurality of rays extending from the image sensor to the periphery of the optically detectable region; and
fitting a circle to the model of the plurality of rays.

20. The system of claim 1, wherein to receive image data from the image sensor comprises receiving a plurality of images from the image sensor, each of the plurality of images including a representation of the first target and a representation of the second target as viewed from different positions.

21. The system of claim 20, wherein the first target comprises a circular optically detectable region, and wherein to determine a spatial feature of the first target comprises:
identifying a periphery of the optically detectable region as represented in each of the plurality of images;
modelling a plurality of rays extending from the image sensor to the periphery of the optically detectable region for each image of the plurality of images; and
fitting a circle to the model of the plurality of rays for the union of the plurality of images.

22.-25. (canceled)

26. The system of claim 1, further comprising a non-transitory processor readable storage medium, wherein to provide the spatial relationship between the second target and the spatial feature of the end effector comprises: providing the spatial relationship between the second target and the spatial feature of the end effector to the non-transitory processor-readable storage medium for storage and subsequent access.

27. The system of claim 1, further comprising a non-transitory processor-readable storage medium having a model of the first target stored thereon, and wherein the processing unit is further configured to receive the model of the first target from the non-transitory processor-readable storage medium, with which to determine the spatial feature of the first target based on the image data.

28. The system of claim 1, further comprising a non-transitory processor-readable storage medium having a model of the second target stored thereon, and wherein the processing unit is further configured to receive the model of the second target from the non-transitory processor-readable storage medium, with which to determine the pose of the second target based on the image data.

29. A computer-implemented method of registering a tool for tracking of said tool, the method comprising:
receiving image data from an image sensor, the image data including:
a representation of a first optically detectable target removably coupled to a first end of the tool with a known spatial relationship to an end effector of the tool, with which to determine a spatial feature of the first target; and
a representation of a second optically detectable target coupled to the tool spatially separate from the first target, with which to determine a pose of the second target;
determining a spatial relationship between the second target and a spatial feature of the end effector, based on a spatial relationship determined between the spatial feature of the first target and the pose of the second target, and based on the known spatial relationship between the first target and the end effector; and
providing the spatial relationship between the second target and the spatial feature of the end effector, for subsequent tracking of the end effector based on the second target.

30. The method of claim 29, wherein the first target comprises a circular optically detectable region, and determining a spatial feature of the first target comprises:
identifying a periphery of the optically detectable region as represented in the image data;
modeling a plurality of rays extending from the image sensor to the periphery of the optically detectable region; and
fitting a circle to the model of the plurality of rays.

31. (canceled)

32. The method of claim 31, wherein receiving image data from the image sensor comprises receiving a plurality of images from the image sensor, each of the plurality of images including a representation of the first target and a representation of the second target, wherein the first target comprises a circular optically detectable region, and wherein determining a spatial feature of the first target comprises:
identifying a periphery of the optically detectable region as represented in each of the plurality of images;
modeling a plurality of rays extending from the image sensor to the periphery of the optically detectable region for each image of the plurality of images; and
fitting a circle to the model of the plurality of rays for the union of the plurality of images.

33.-51. (canceled)
